(19) United States
(12) Patent Application Publication
(10) Pub. No.: US 2010/0311946 A1
(43) Pub. Date: Dec. 9, 2010

(54) Solid Phase Peptide for the Production of Goserelin

(75) Inventors: Kripa S. Srivastava, Chesterfield, MO (US); Matthew R. Davis, St. Louis, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/787,447

(22) Filed: May 26, 2010

Related U.S. Application Data
(60) Provisional application No. 61/183,602, filed on Jun. 3, 2009.

(51) Int. Cl. C07K 7/06

(52) U.S. Cl. 530/328

(57) ABSTRACT

The present invention provides a process for the production of goserelin. In particular, the process of the invention allows the use of side chain protecting groups during synthesis of the peptide, and the addition of the azaglycine moiety of the peptide.

### CROSS REFERENCE TO RELATED APPLICATIONS

This application claims the benefit of U.S. Provisional Application No. 61/183,602 filed Jun. 3, 2009, which is incorporated herein in its entirety.

### FIELD OF THE INVENTION

The invention provides a process for the production of goserelin using solid phase peptide synthesis.

### BACKGROUND OF THE INVENTION

Goserelin is a synthetic decapeptide and an analog of the naturally occurring hormone Leuteinizing Hormone-Releasing Hormone (LH-RH) or Gonadotropin Releasing Hormone (GnRH). Its chemical structure is p.Glu-His-Trp-Ser-Tyr-D.Ser(tBu)-Leu-Arg-Pro-Azagly-CONH2, where p.Glu is pyroglutamic acid, tBu is a tertiary butyl group covalently attached to the side chain of the D.serine amino acid, and azagly is a glycine analog where the alpha-C of the glycine is replaced with a nitrogen. It is chemically described as an acetate salt of [D.Ser(tBu)⁶-Azagly¹⁰]LH-RH. Goserelin is used to treat hormone-sensitive cancers of the prostate and breast and some benign gynecological disorders such as endometriosis, uterine fibroids and endometrial thinning. Goserelin is a unique molecule compared to other peptides. Two features of this structure are incompatible with traditional solid phase peptide synthesis routes. The first is the unusual azagly C-terminal amino acid, which is incompatible with traditional methods for linking amino acids to solid supports. The second feature of the molecule is the t.butyl side chain protecting group linked to the D.serine moiety. Because this side chain protecting group is retained in the final product, most methods to produce goserelin devised to date use amino acids without side chain protection. In addition, retaining this group during release of the completed peptide by the use of traditional methods is also difficult. Because of these two uncommon features, synthesis of goserelin has consistently been difficult, resulting in the production of only modest goserelin yields. In addition, existing methods of goserelin synthesis often fail to produce the desired peptide in sufficient yield and purity.

Therefore, there is a need in the art for a facile and economical method of synthesis of goserelin that would allow the use of side chain protecting groups during synthesis of the peptide, and the addition of the azaglycine moiety of the peptide.

### SUMMARY OF THE INVENTION

Briefly, therefore, the present invention provides a process for solid phase synthesis of goserelin using amino acids with protected side chains. In one aspect, the invention encompasses a process for solid phase synthesis of goserelin, the process comprising:
a. providing a solid support coupled with azaglycine;
b. activating the carboxy group of a proline residue that has its amine protected by a Fmoc group or a Boc group, followed by coupling the proline residue to the azaglycine residue on the solid support of (a), and treatment of the solid support with an agent to deprotect the amine group of the proline residue;
c. activating the carboxy group of an arginine residue that has its amine protected by a Fmoc group or a Boc group, followed by coupling the arginine residue to the proline residue on the solid support of (b), and treatment of the solid support with an agent to deprotect the amine group of the arginine residue;
d. activating the carboxy group of a leucine residue that has its amine protected by a Fmoc group or Boc group, followed by coupling the leucine residue to the arginine residue on the solid support of (c), and treatment of the solid support with an agent to deprotect the amine group of the leucine residue;
e. activating the carboxy group of a D-serine residue that has its amine protected by a Fmoc group and its side chain protected by a tertiary butyl alkyl group, followed by coupling the D-serine residue to the leucine residue on the solid support of (d), and treatment of the solid support with a base to deprotect the amine group of the D-serine residue;
f. activating the carboxy group of a tyrosine residue that has its amine protected by a Fmoc group and its side chain protected by an acid labile group, followed by coupling the tyrosine residue to the D-serine residue on the solid support of (e), and treatment of the solid support with a base to deprotect the amine group of the tyrosine residue;
g. activating the carboxy group of a serine residue that has its amine protected by a Fmoc group and its side chain protected by an acid labile group, followed by coupling the serine residue to the tyrosine residue on the solid support of (f), and treatment of the solid support with a base to deprotect the amine group of the serine residue;
h. activating the carboxy group of a tryptophan residue that has its amine protected by a Fmoc group, followed by coupling the tryptophan residue to the serine residue on the solid support of (g), and treatment of the solid support with a base to deprotect the amine group of the tryptophan residue;
i. activating the carboxy group of a histidine residue that has its amine protected by a Fmoc group and its side chain protected by an acid labile group, followed by coupling the histidine residue to the to the tryptophan residue on the solid support of (h), and treatment of the solid support with a base to deprotect the amine group of the histidine residue;
j. activating the carboxy group of a pyroglutamic acid residue, followed by coupling the pyroglutamic acid residue to the histidine residue on the solid support of (i); and
k. simultaneously contacting the solid support of (j) with an acid in a manner such that goserelin is released from the solid support and the side chains of tyrosine, serine, and histidine are deprotected, but the side chain of D-serine remains protected with tertiary butyl alkyl.

In another iteration, the invention encompasses a process for solid phase synthesis of goserelin, the process comprising:
a. providing a solid support coupled with a proline residue;
b. activating the carboxy group of an arginine residue that has its amine protected by a Boc group or Fmoc group, followed by coupling the arginine residue to the proline residue on the solid support of (a), and treatment of the solid support with an agent to deprotect the amine group of the arginine residue;
c. activating the carboxy group of a leucine residue that has its amine protected by a Boc group or Fmoc group, followed by coupling the leucine residue to the arginine residue on the solid support of (b), and treatment of the solid support with an agent to deprotect the amine group of the leucine residue;
d. activating the carboxy group of a D-serine residue that has its amine protected by a Fmoc group and its side chain protected by a tertiary butyl alkyl group, followed by coupling the D-serine residue to the leucine residue on the solid support of (c), and treatment of the solid support with a base to deprotect the amine group of the D-serine residue;
e. activating the carboxy group of a tyrosine residue that has its amine protected by a Fmoc group and its side chain protected by an acid labile group, followed by coupling the tyrosine residue to the D-serine residue on the solid support of (d), and treatment of the solid support with a base to deprotect the amine group of the tyrosine residue;
f. activating the carboxy group of a serine residue that has its amine protected by a Fmoc group and its side chain protected by an acid labile group, followed by coupling the serine residue to the tyrosine residue on the solid support of (e), and treatment of the solid support with a base to deprotect the amine group of the serine residue;
g. activating the carboxy group of a tryptophan residue that has its amine protected by a Fmoc group, followed by coupling the tryptophan residue to the serine residue on the solid support of (f), and treatment of the solid support with a base to deprotect the amine group of the tryptophan residue;
h. activating the carboxy group of a histidine residue that has its amine protected by a Fmoc group and its side chain protected by an acid labile group, followed by coupling the histidine residue to the to the tryptophan residue on the solid support of (g), and treatment of the solid support with a base to deprotect the amine group of the histidine residue;
i. activating the carboxy group of a pyroglutamic acid residue, followed by coupling the pyroglutamic acid residue to the histidine residue on the solid support of (h);
j. contacting the solid support of (j) with hydrazine in a manner such that a peptide hydrazide is released from the solid support, and the side chains of D-serine, tyrosine, serine, and histidine remain protected;
k. contacting the peptide hydrazide of (k) with an acid in a manner such that the side chains of tyrosine, serine, and histidine are deprotected, but the side chain of D-serine remains protected with tertiary butyl alkyl; and
l. contacting the peptide hydrazide from (k) with a cyanate ion source to form goserelin.

In an additional aspect, the invention encompasses a process for solid phase synthesis of goserelin, the process comprising:
a. providing a solid support coupled with a proline residue;
b. activating the carboxy group of an arginine residue that has its amine protected by a Boc group or Fmoc group, followed by coupling the arginine residue to the proline residue on the solid support of (a), and treatment of the solid support with an agent to deprotect the amine group of the arginine residue;
c. activating the carboxy group of a leucine residue that has its amine protected by a Boc group or Fmoc group, followed by coupling the leucine residue to the arginine residue on the solid support of (b), and treatment of the solid support with an agent to deprotect the amine group of the leucine residue;
d. activating the carboxy group of a D-serine residue that has its amine protected by a Fmoc group and its side chain protected by a tertiary butyl alkyl group, followed by coupling the D-serine residue to the leucine residue on the solid support of (c), and treatment of the solid support with a base to deprotect the amine group of the D-serine residue;
e. activating the carboxy group of a tyrosine residue that has its amine protected by a Fmoc group and its side chain protected by an acid labile group, followed by coupling the tyrosine residue to the D-serine residue on the solid support of (d), and treatment of the solid support with a base to deprotect the amine group of the tyrosine residue;
f. activating the carboxy group of a serine residue that has its amine protected by a Fmoc group and its side chain protected by an acid labile group, followed by coupling the serine residue to the tyrosine residue on the solid support of (e), and treatment of the solid support with a base to deprotect the amine group of the serine residue;
g. activating the carboxy group of a tryptophan residue that has its amine protected by a Fmoc group, followed by coupling the tryptophan residue to the serine residue on the solid support of (f), and treatment of the solid support with a base to deprotect the amine group of the tryptophan residue;
h. activating the carboxy group of a histidine residue that has its amine protected by a Fmoc group and its side chain protected by an acid labile group, followed by coupling the histidine residue to the to the tryptophan residue on the solid support of (g), and treatment of the solid support with a base to deprotect the amine group of the histidine residue;
i. activating the carboxy group of a pyroglutamic acid residue, followed by coupling the pyroglutamic acid residue to the histidine residue on the solid support of (h);
j. contacting the solid support of (i) with a cleaving agent in a manner such that a peptide acid is cleaved from the solid support, and the side chains of D-serine, tyrosine, serine, and histidine remain protected;
k. contacting the peptide acid of (j) with hydrazine to form a peptide hydrazide, in a manner such that a peptide acid is cleaved from the solid support, and the side chains of D-serine, tyrosine, serine, and histidine remain protected;
l. contacting the peptide hydrazide of (k) with an acid in a manner such that the side chains of tyrosine, serine, and histidine are deprotected, but the side chain of D-serine remains protected with tertiary butyl alkyl; and
m. contacting the peptide hydrazide from (l) with a cyanate ion source to form goserelin.

Other aspects and iterations of the invention are described in more detail below.

### DETAILED DESCRIPTION OF THE INVENTION

A process that simplifies solid phase synthesis of goserelin has been developed. The process simplifies the addition of the azaglycine amino acid analog, and allows the use of amino acid residues with side chain protection groups during peptide synthesis. The process allows for the retention of the t-butyl side chain protection group during cleavage of the peptide from the resin and deprotection of the peptide. As illustrated in the examples, the process of the invention generally produces goserelin in higher yield and purity compared to other methods currently used to synthesize goserelin.

### (I) Synthesis of Goserelin by Simultaneous Release and Deprotection of Peptide

In one aspect of the invention, the peptide may be synthesized in accordance with the diagram below. In essence, a solid support coupled with azaglycine is provided. This is followed by activating the carboxy group of a proline residue that has its amine protected by a Fmoc group or a Boc group, followed by coupling the proline residue to the azaglycine residue on the solid support, and treatment of the solid support with an agent to deprotect the amine group of the proline residue. This process is repeated for an arginine residue that has its amine protected by a Fmoc group or a Boc group, a leucine residue that has its amine protected by a Fmoc group or Boc group, a D-serine residue that has its amine

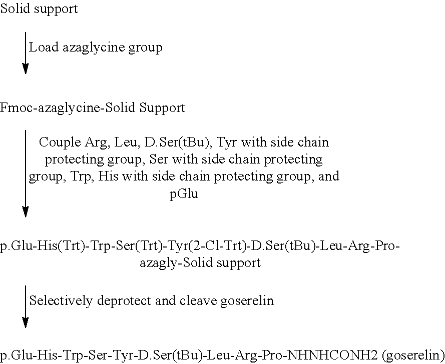

protected by a Fmoc group and its side chain protected by a tertiary butyl alkyl group, a tyrosine residue that has its amine protected by a Fmoc group and its side chain protected by an acid labile group, a serine residue that has its amine protected by a Fmoc group and its side chain protected by an acid labile group, a tryptophan residue that has its amine protected by a Fmoc group, a histidine residue that has its amine protected by a Fmoc group and its side chain protected by an acid labile group, and a pyroglutamic acid residue. The resulting decapeptide attached to the solid support is simultaneously contacted with an acid in a manner such that goserelin is released from the solid support and the side chains of tyrosine, serine, and histidine are deprotected, but the side chain of D-serine remains protected with tertiary butyl alkyl.
(A) Solid Support Coupled with Azaglycine:

In essence, a solid support coupled with azaglycine is first provided, Generally, the solid support comprises an amide group that will become part of the peptide upon cleavage to produce a peptide amide. A non-limiting list of suitable solid supports that may be used in the preparation of peptide amides may include NovaSyn® TGR resin, Rink amide resin, Rink amid MBHA resin, Rink amide AM resin, Rink amide PEGA resin, Rink amide NovaGel® resin, Sieber amide resin, and NovaSyn® TG Sieber resin. In a preferred embodiment, the solid support is Sieber amide resin. As is commonly known in the art, an Fmoc group may be covalently attached to the solid support. If an Fmoc group is covalently attached to the solid support, the Fmoc group may be removed using methods described further below.

The solid support is coupled with an azaglycine moiety. In some embodiments, the azaglycine moiety may be loaded onto the solid support by first synthesizing an N¹-Fluoren-9-ylmethoxycarbonyl-N²-succinimido-oxycarbonylhydrazine (Fmoc-Azagly-OSu) in accordance with Reaction Scheme 1. Referring to Reaction Scheme 1, hydrazine is first reacted with Fmoc-succinimido carbonate (Fmoc-OSu) to produce Fmoc hydrazine. Fmoc-hydrazine is then reacted with disuccinimidyl carbonate to produce Fmoc-Azagly-OSu.

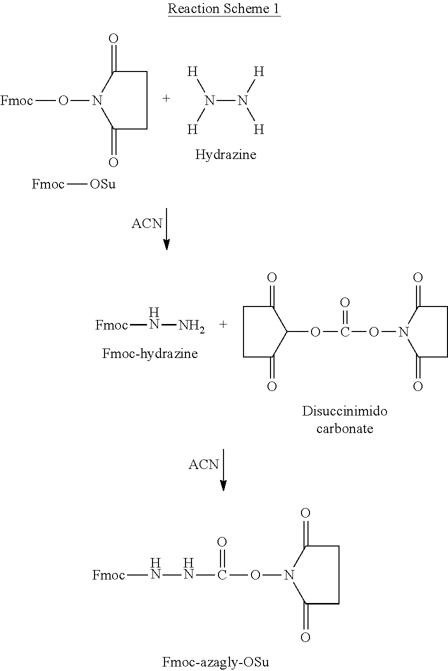

Typically, hydrazine is reacted with Fmoc-OSu in the presence of an aprotic solvent. For example, suitable solvents include, but are not limited to, acetone, acetonitrile, diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), 1,4-dioxane, N-methyl-2-pyrrolidinone (NMP), ethyl acetate, ethyl formate, ethyl methyl ketone, formamide, hexachioroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyl tetrahydrofuran, trichloromethane, and combinations thereof. Generally speaking, the amount of Fmoc-OSu to the amount of hydrazine may be expressed as a molar ratio of from about 1:0.5 to about 1:2. In an exemplary embodiment, production of Fmoc-hydrazine is carried out in the presence of acetonitrile at approximately room temperature with the amount of Fmoc-OSu to the amount of hydrazine is a molar ratio of about 1:1.

Fmoc-hydrazine is typically reacted with disuccinimidyl carbonate in the presence of an aprotic solvent. The amount of Fmoc-hydrazine to the amount of disuccinimidyl carbonate may be expressed as a molar ratio of from about 1:0.5 to about 1:2. In an exemplary embodiment, Fmoc-hydrazine is reacted with disuccinimidyl carbonate in the presence of acetonitrile at approximately room temperature with the amount of Fmoc-hydrazine to the amount of disuccinimidyl carbonate is a molar ratio of about 1:1,

Fmoc-azagly-Osu is reacted with the solid support in the presence of an aprotic solvent. In preferred embodiments, the Fmoc-azagly-Osu is reacted with the solid support in the presence of DMF. The molar ratio of the Fmoc-azagly-Osu to the solid support may range from about 4:1 to about 1:1. In one embodiment, the molar ratio of the Fmoc-azagly-Osu to the solid support may be about 2:1.

In other embodiments, the solid support may be loaded with Fmoc-azagly by reacting the solid support with Fmoc-hydrazine and triphosgene in the presence of diisopropylethylamine (DIEA) activating compound in the presence of an aprotic solvent. In a preferred embodiment, the aprotic solvent is DMF. The amount of the various reactants in the reaction can and will vary. Typically, the molar ratio of the solid support to Fmoc-azagly to triphosgene may range from about 1:1:1 to about 1:3:3. In one embodiment, the molar ratio of the solid support to Fmoc-azagly to triphosgene may be about 5:1:0.1. The reaction conditions for loading the solid support with Fmoc-azagly using triphosgene, such as reaction time and temperature may also vary without departing from the scope of the invention. By way of non-limiting example, the reaction time may range from several hours to several days, and the reaction temperature may range from approximately room temperature to about 0° C. Exemplary reaction parameters of the process are detailed in the examples.

Upon loading the Fmoc-Azagly onto the solid support, the Fmoc group may be removed using methods described below for Fmoc chemistry.

### (B) Peptide Synthesis:

As detailed herein, after the solid support is loaded with the azagly moiety, peptide elongation may be conducted using methods of solid phase peptide synthesis known in the art. In general, solid phase peptide synthesis methods known in the art involve the sequential coupling of amino acids that have their amines protected. After each coupling step, the terminal amino acid protecting group is then cleaved to provide a free amine group ready for coupling the next amino acid in the next addition cycle. Commonly used amine protecting groups may include tert-Butoxycarbonyl (Boc) and 9H-fluoren-9-yl-methoxycarbonyl (Fmoc) protecting groups.

Accordingly, the synthesis of goserelin involves the following steps: activating the carboxy group of a praline residue that has its amine protected by a Fmoc group or a Boc group, followed by coupling the proline residue to the azaglycine residue on the solid support, and treatment of the solid support with an agent to deprotect the amine group of the proline residue; activating the carboxy group of an arginine residue that has its amine protected by a Fmoc group or a Boc group, followed by coupling the arginine residue to the proline residue, and treatment of the solid support with an agent to deprotect the amine group of the arginine residue; activating the carboxy group of a leucine residue that has its amine protected by a Fmoc group or Boc group, followed by coupling the leucine residue to the arginine residue, and treatment of the solid support with an agent to deprotect the amine group of the leucine residue; activating the carboxy group of a D-serine residue that has its amine protected by a Fmoc group and its side chain protected by a tertiary butyl alkyl group, followed by coupling the D-serine residue to the leucine residue, and treatment of the solid support with a base to deprotect the amine group of the D-serine residue; activating the carboxy group of a tyrosine residue that has its amine protected by a Fmoc group and its side chain protected by an acid labile group, followed by coupling the tyrosine residue to the D-serine residue, and treatment of the solid support with a base to deprotect the amine group of the tyrosine residue; activating the carboxy group of a serine residue that has its amine protected by a Fmoc group and its side chain protected by an acid labile group, followed by coupling the serine residue to the tyrosine residue, and treatment of the solid support with a base to deprotect the amine group of the serine residue; activating the carboxy group of a tryptophan residue that has its amine protected by a Fmoc group, followed by coupling the tryptophan residue to the serine residue, and treatment of the solid support with a base to deprotect the amine group of the tryptophan residue; activating the carboxy group of a histidine residue that has its amine protected by a Fmoc group and its side chain protected by an acid labile group, followed by coupling the histidine residue to the to the tryptophan residue, and treatment of the solid support with a base to deprotect the amine group of the histidine residue; activating the carboxy group of a pyroglutamic acid residue, followed by coupling the pyroglutamic acid residue to the histidine residue.

In one embodiment, the proline residue has its amine protected by a Boc group, the arginine residue has its amine protected by a Boc group, and the leucine residue has its amine protected by a Boc group. In another embodiment, the proline residue has its amine protected by a Boo group, the arginine residue has its amine protected by a Boc group, and the leucine residue has its amine protected by a Fmoc group. In another embodiment, the proline residue has its amine protected by a Boc group, the arginine residue has its amine protected by a Fmoc group, and the leucine residue has its amine protected by a Boc group. In yet another embodiment, the proline residue has its amine protected by a Fmoc group, the arginine residue has its amine protected by a Boc group, and the leucine residue has its amine protected by a Boc group. In another embodiment, the proline residue has its amine protected by a Boc group, the arginine residue has its amine protected by a Fmoc group, and the leucine residue has its amine protected by a Fmoc group. In an additional embodiment, the proline residue has its amine protected by a Fmoc group, the arginine residue has its amine protected by a Boc group, and the leucine residue has its amine protected by a Fmoc group. In yet another embodiment, the proline residue has its amine protected by a Fmoc group, the arginine residue has its amine protected by a Fmoc group, and the leucine residue has its amine protected by a Boc group. In a preferred embodiment, the proline residue has its amine protected by a Fmoc group, the arginine residue has its amine protected by a Fmoc group, and the leucine residue has its amine protected by a Fmoc group.

Amine groups protected with Fmoc may be deprotected by treatment with an organic base. Suitable organic bases include piperidine, cyclohexylamine, 1,5-diazabicyclo [5,4,0]undec-5-ene, ethanolamine, pyrrolidine 1,8-diazabicyclo[5.4.0]undec-7-ene, diethylamine, morpholine, and mixtures thereof. In an exemplary embodiment, the base is piperidine. Typically, the amount of organic base used in Fmoc deprotection when the base is piperidine will range from about 5% to about 50% (v/v).

The Fmoc deprotection reaction is carried out in the presence of a solvent at approximately room temperature. Non-limiting examples of suitable solvents include anisole, dimethylformamide, dimethylsulfoxide, dimethyl acetamide, dichloromethane, N-methyl pyrrolidinone, and mixtures thereof. A list of additional suitable solvents can be found in Tetrahedron Letters 39:8451-54 (1998), which is incorporated herein by reference in its entirety.

Amine groups protected with Boc may be deprotected by treatment with an acid. Suitable acids may include, but are not limited to, trifluoroacetic acid (TFA) and hydrochloric acid (HCl). In a preferred embodiment, the acid is TFA. Typically, the amount of acid used in Boc deprotection when the acid is TFA will range from about 40% to about 60% (v/v). In a preferred embodiment, the amount of acid used in Boc deprotection when the acid is TFA may be about 50% (v/v).

The Boc deprotection reaction is carried out in the presence of a solvent at approximately room temperature. Boc deprotection is typically carried out in the presence of an organic solvent. For example, suitable solvents include, but are not limited to, alkane and substituted alkane solvents (including cycloalkanes), aromatic hydrocarbons, esters, ethers, ketones, combinations thereof, and the like. Specific organic solvents that may be employed, include, for example, acetonitrile, benzene, butyl acetate, t-butyl methylketone, chlorobenzene, chloroform, chloromethane, cyclohexane, dichloromethane, dichloroethane, dichloroethene, fluorobenzene, heptane, hexane, isobutylmethylketone, isopropyl acetate, methylethylketone, methyltetrahydrofuran, pentyl acetate, n propyl acetate, tetrahydrofuran, toluene, and combinations thereof. In an exemplary embodiment, Boo deprotection is carried out in the presence of dichloromethane.

For the coupling reaction, the carboxyl group of the incoming amino acid is usually activated. Suitable activating compounds include carbodiimides, or those belonging to the aromatic oximes class or combinations thereof. In one embodiment, the carbodiimide is selected from dicyclohexylcarboimide (DCC), DIEA, or diisopropylcarboimide (DIC). In another embodiment, the aromatic oxime is selected from 1-hydroxy-benzotriazole(HOBt), and 1-hydroxy-7-aza-benzotriazole (HOAt). In an exemplary embodiment, the activating compounds are DIC and HOBt. Other suitable activating compounds include HATU/HOAT, PyBOP/HOBT, or OPFP preactivated amino acids/HOBT.

The amount of the various reactants in the coupling reaction can and will vary greatly. Typically the molar ratio of the solid support to the Bac- or Fmoc-amino acid to the activating compound will range from about 1:1:1 to about 1:5:5. In one embodiment, the molar ratio of the solid support to the Boc- or Fmoc-amino acid to the activating compound may be about 1:1.5:1.5.

The progress of amino acid couplings may be followed using a ninhydrin reaction, as described in the examples. The ninhydrin solution turns dark blue (positive result) in the presence of a free primary amine but is otherwise colorless (negative result).

Acid-labile side chain protecting groups generally protect the side chains of the tyrosine, serine, and histidine amino acids. As a general rule, side chain protecting groups should be labile under conditions that would allow the deprotection of the tyrosine, serine, and histidine residues, but maintain the tBu side chain protecting group of D.Ser. As such, the acid-labile protecting groups for histidine may be selected from the group consisting of methyltrityl, methoxytrityl, or trityl. In a preferred embodiment, the acid-labile protecting group for histidine is trityl. The acid-labile protecting groups for tyrosine may be selected from the group consisting of trityl or chlorotrityl. In a preferred embodiment, the acid-labile protecting group for tyrosine is chlorotrityl. The acid-labile protecting groups for serine may be selected from the group consisting of trityl or methyltrityl for serine. In a preferred embodiment, the acid-labile protecting group for serine is trityl.

(C) Release of Goserelin from Solid Support:

Once the final amino acid (p.Glu) has been added, the solid support from Section I(C) above may be contacted with an acid in such a manner that the peptide is released from the solid support and the side chains of tyrosine, serine, and histidine are deprotected, but the side chain of D-serine remains protected with tertiary butyl alkyl, thus producing goserelin. The acid may be selected from the group consisting of acetic acid (AcOH), TFA, hydrochloric acid (HCl), and trifluoroethanol (TFE) or combinations thereof. In general, the solid support will be treated with trifluoroacetic acid (TFA) in the presence of appropriate scavengers in an organic solvent. The amount of TFA typically used for cleavage of the protected peptide from the solid support may range from about 1% to about 15% (v/v) in an organic solvent. More typically, the amount of TFA used for cleavage of the protected peptide from the solid support may range from about 5% to about 10% (v/v) in an organic solvent. In a preferred embodiment, the amount of TFA used to release the protected peptide from the solid support is 8% (v/v) in dichloromethane. Scavengers that may be used to release the peptide may include phenol, water, 1,2-ethanedithiol, and triisopropylsilane (TIS). In a preferred embodiment, the scavenger is TIS. The amount of TIS typically used for cleavage of the protected peptide from the solid support may range from about 1% to about 10% (v/v). In a preferred embodiment, the amount of TIS used to release the protected peptide from the solid support is 5% (v/v). In an exemplary embodiment, the amount of TFA used to release the protected peptide from the solid support is 8% (v/v), and the amount of TIS is 5% (v/v), in dichloromethane.

Goserelin is typically analyzed by chromatography, such as reverse phase HPLC or mass spectrometry after it is cleaved from the solid support. As will be appreciated by a skilled artisan the yield and purity can and will vary depending upon the peptide produced. The yield will generally range from about 40% to greater than about 90%. More typically, the yield will range from about 60% to greater than about 80%. The purity will generally range from about 65% to greater than about 99% as determined by HPLC.

### (II) Synthesis of Goserelin by Adding Azaglycine After Synthesis of the Nonapeptide

In other aspects of the invention, the peptide may be synthesized in accordance with the diagram below. In essence, a solid support coupled with a proline residue is first provided. This is followed by activating the carboxy group of an arginine residue that has its amine protected by a Fmoc group or a Boc group, followed by coupling the arginine residue to the proline residue on the solid support, and treatment of the solid support with an agent to deprotect the amine group of the arginine residue. This process is repeated for a leucine residue that has its amine protected by a Fmoc group or Boc group, a D-serine residue that has its amine protected by a Fmoc group and its side chain protected by a tertiary butyl alkyl group, a tyrosine residue that has its amine protected by a Fmoc group and its side chain protected by an acid labile group, a serine residue that has its amine protected by a Fmoc group and its side chain protected by an acid labile group, a tryptophan residue that has its amine protected by a Fmoc group, a histidine residue that has its amine protected by a Fmoc group and its side chain protected by an acid labile group, and a pyroglutamic acid residue. Azaglycine is then added to the resulting nonapeptide and the side chains of tyrosine, serine, and histidine are deprotected, in a manner such that the side chain of D-serine remains protected with tertiary butyl alkyl, to form goserelin.

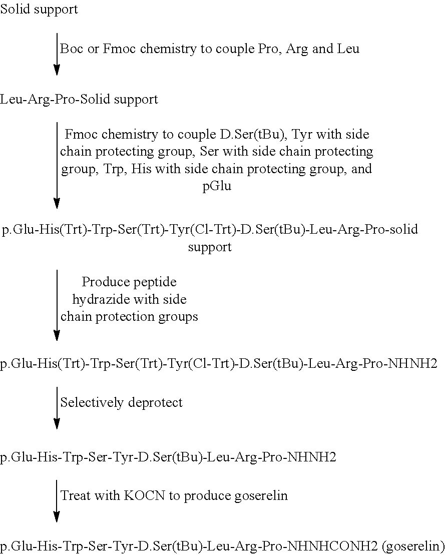

(A) Solid Support Coupled with a Proline Residue:

In essence, a solid support coupled with a proline residue is first provided. The solid support of the invention may be any solid support that may be used in the preparation of peptide acids. Non-limiting examples of suitable solid supports that may be used in the preparation of peptide acids may include chlorotrityl resin, trityl resin, methyltrityl resins, methoxytrityl resins, NovaSyn® TGT resin, HMPB-AM resin, HMPB-BHA resin, HMPB-MBHA resin, Wang resin, NovaSyn-TGA resin, HMPA-PEGA resin, HMPA-NovaGel resin, PAM resin, and Merrifield resin. In one embodiment, the solid support may be 2-chlorotrityl chloride resin. In another embodiment, the solid support may be Merrifield resin.

The proline residue coupled to the solid support may be protected with a Boc- or Fmoc-protecting group. In one embodiment, the solid support is coupled with a proline residue with its amine protected with a Boc protecting group. Methods of loading the first Boc-protected amino acid are known to those skilled in the art and can be found in, for example, Solid Phase Peptide Synthesis, Academic Press (1997), which is incorporated herein by reference in its entirety.

In another embodiment, the solid support is coupled with a praline residue with its amine protected with an Fmoc protecting group. The Fmoc-protected proline residue may be coupled to the solid support by methods known in the art. Methods of loading the first Fmoc-protected amino acid are known to those skilled in the art and can be found in, for example, Fmoc Solid Phase Peptide Synthesis: A Practical Approach (Practical Approach Series) Oxford University Press, USA; 1 edition (Mar. 2, 2000), which is incorporated herein by reference in its entirety. Non-limiting examples of methods for attaching the first amino acid to the solid support include the symmetrical anhydride method, the dichlorobenzoyl chloride method, DIC-HOBt method, and the MSNT/Melm method.

Upon loading the Fmoc-Pro onto the solid support, the Fmoc group may be removed using methods described in Section (IB) above.

### (B) Peptide Synthesis:

As detailed herein, after the solid support is loaded with the praline residue, peptide elongation may be conducted using methods of solid phase peptide synthesis as described in Section (IB) above. Accordingly, in this aspect of the invention, the synthesis involves the following steps: activating the carboxy group of an arginine residue that has its amine protected by a Fmoc group or a Boc group, followed by coupling the arginine residue to the proline residue, and treatment of the solid support with an agent to deprotect the amine group of the arginine residue; activating the carboxy group of a leucine residue that has its amine protected by a Fmoc group or Boc group, followed by coupling the leucine residue to the arginine residue, and treatment of the solid support with an agent to deprotect the amine group of the leucine residue; activating the carboxy group of a D-serine residue that has its amine protected by a Fmoc group and its side chain protected by a tertiary butyl alkyl group, followed by coupling the D-serine residue to the leucine residue, and treatment of the solid support with a base to deprotect the amine group of the D-serine residue; activating the carboxy group of a tyrosine residue that has its amine protected by a Fmoc group and its side chain protected by an acid labile group, followed by coupling the tyrosine residue to the D-serine residue, and treatment of the solid support with a base to deprotect the amine group of the tyrosine residue; activating the carboxy group of a serine residue that has its amine protected by a Fmoc group and its side chain protected by an acid labile group, followed by coupling the serine residue to the tyrosine residue, and treatment of the solid support with a base to deprotect the amine group of the serine residue; activating the carboxy group of a tryptophan residue that has its amine protected by a Fmoc group, followed by coupling the tryptophan residue to the serine residue, and treatment of the solid support with a base to deprotect the amine group of the tryptophan residue; activating the carboxy group of a histidine residue that has its amine protected by a Fmoc group and its side chain protected by an acid labile group, followed by coupling the histidine residue to the to the tryptophan residue, and treatment of the solid support with a base to deprotect the amine group of the histidine residue; activating the carboxy group of a pyroglutamic acid residue, followed by coupling the pyroglutamic acid residue to the histidine residue.

In one embodiment, the proline residue has its amine protected by a Fmoc group, the arginine residue has its amine protected by a Fmoc group, and the leucine residue has its amine protected by a Fmoc group. In another embodiment, the praline residue has its amine protected by a Boc group, the arginine residue has its amine protected by a Boc group, and the leucine residue has its amine protected by a Fmoc group. In another embodiment, the proline residue has its amine protected by a Boc group, the arginine residue has its amine protected by a Fmoc group, and the leucine residue has its amine protected by a Boc group. In yet another embodiment, the proline residue has its amine protected by a Fmoc group, the arginine residue has its amine protected by a Boc group, and the leucine residue has its amine protected by a Boc group. In another embodiment, the proline residue has its amine protected by a Boc group, the arginine residue has its amine protected by a Fmoc group, and the leucine residue has its amine protected by a Fmoc group. In an additional embodiment, the proline residue has its amine protected by a Fmoc group, the arginine residue has its amine protected by a Bac group, and the leucine residue has its amine protected by a Fmoc group. In yet another embodiment, the proline residue has its amine protected by a Fmoc group, the arginine residue has its amine protected by a Fmoc group, and the leucine residue has its amine protected by a Boc group. In a preferred embodiment, the praline residue has its amine protected by a Boc group, the arginine residue has its amine protected by a Boc group, and the leucine residue has its amine protected by a Boc group.

Acid-labile protecting groups generally protect the side chains of the tyrosine, serine, and histidine amino acids. The acid-labile protecting groups for histidine may be selected from the group consisting of methyltrityl, methoxytrityl, or trityl. In a preferred embodiment, the acid-labile protecting group for histidine is trityl. The acid-labile protecting groups for tyrosine may be selected from the group consisting of trityl or chlorotrityl. In a preferred embodiment, the acid-labile protecting group for tyrosine is chlorotrityl. The acid-labile protecting groups for serine may be selected from the group consisting of trityl or methyltrityl for serine. In one preferred embodiment, the acid-labile protecting group for serine is trityl. In another preferred embodiment, the acid-labile protecting group for serine is methyltrityl.

(C) Release of Peptide from the Solid Support:

### (a) Release of Peptide Hydrazide.

In one aspect of the invention, once the final amino acid (p.Glu) has been added, the peptide from Section II(B) above may be released from the solid support by contacting the peptide-solid support with hydrazine in a manner such that a peptide hydrazide is released from the solid support and the side chains of D-serine, tyrosine, serine, and histidine remain protected.

In general, the peptide-solid support is contacted with hydrazine in the presence of an aprotic solvent, a protic solvent or a combination of aprotic and protic solvents. Suitable examples of aprotic solvents include diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), 1,4-dioxane, N-methyl-2-pyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyl tetrahydrofuran, toluene, trichloromethane, and combinations thereof. Suitable examples of protic solvents include, but are not limited to, methanol, ethanol, isopropanol, n-propanol, isobutanol, n-butanol, s-butanol, t-butanol, formic acid, acetic acid, and combinations thereof. In a preferred embodiment, the peptide-solid support is contacted with hydrazine in a combination of aprotic and protic solvents. In one embodiment, the peptide-solid support is contacted with hydrazine in dimethylformamide. The amount of dimethylformamide to the amount of hydrazine may be expressed as a volume ratio of about 1:1 to about 1:8. In another embodiment, the peptide-solid support is contacted with hydrazine in a combination of dimethylformamide and methanol. The amount of dimethylformamide to the amount of methanol to the amount of hydrazine may be expressed as a volume ratio of about 1:1:1 to about 1:1:2.

The reaction conditions for producing a peptide hydrazide, such as reaction time, and temperature may vary without departing from the scope of the invention. By way of non-limiting example, the reaction time may range from several hours to several days, and the reaction temperature may range from about 0° C. to approximately room temperature. Exemplary reaction parameters are detailed in the examples.

### (b) Release of Peptide Acid and Conversion of Peptide Acid to Peptide Hydrazide.

In another aspect of the invention, once the final amino acid (p.Glu) has been added, the peptide from Section (IIB) above may be released from the solid support by contacting the peptide-solid support with a cleaving agent in a manner such that a peptide acid is released from the solid support and the side chains of D-serine, tyrosine, serine, and histidine remain protected. Next, the peptide acid may be contacted with hydrazine to form a peptide hydrazide, in a manner such that a peptide acid is cleaved from the solid support, and the side chains of D-serine, tyrosine, serine, and histidine remain protected.

In general, the peptide-solid support is contacted with a cleaving agent. The cleaving agent may be selected from the group consisting of an acid, trifluoroethanol (TFE) or combinations thereof. Non-limiting examples of acids that may be suitable for cleaving the peptide acid from the solid support include acetic acid (AcOH), TFA, and hydrochloric acid (HCl). In a preferred embodiment, the peptide-solid support is contacted with a 3:7 (v:v) ratio of TFE in DCM. The reaction conditions for producing a peptide acid, such as reaction time, and temperature may vary without departing from the scope of the invention. By way of non-limiting example, the reaction time may range from several hours to several days, and the reaction temperature may range from about 0° C. to approximately room temperature.

### (D) Deprotection of Peptide Hydrazide.

The peptide hydrazide from any aspect of Section (IIC) above is contacted with an acid in a manner such that the side chains of tyrosine, serine, and histidine are deprotected, but the side chain of D-serine remains protected with tertiary butyl alkyl. In general, the solid support may be treated with trifluoroacetic acid (TFA) in the presence of appropriate scavengers in an organic solvent. The amount of TFA typically used for cleavage of the protected peptide from the solid support may range from about 0.5% to about 10% (v/v) in an organic solvent. More typically, the amount of TFA used for cleavage of the protected peptide from the solid support may range from about 1% to about 5% (v/v) in an organic solvent. Scavengers that may be used to release the peptide may include phenol, water, 1,2-ethanedithiol, and triisopropylsilane (TIS). In a preferred embodiment, the scavenger is TIS. The amount of TIS typically used for cleavage of the protected peptide from the solid support may range from about 1% to about 10% (v/v). In an exemplary embodiment, the amount of TFA used to release the protected peptide from the solid support is 2% (v/v), and the amount of TIS is 5% (v/v), in dichloromethane. In another exemplary embodiment, the amount of TFA used to release the protected peptide from the solid support is 3% (v/v), and the amount of TIS is 3% (v/v), in dichloromethane.

### (E) Production of Goserelin.

After the peptide hydrazide is deprotected, it is contacted with a cyanate ion source to form goserelin. Suitable cyanate ions may be provided by an alkali metal cyanate. Non-limiting examples of alkali metal cyanates may include potassium cyanate, methyl cyanate, or sodium cyanate. In a preferred embodiment, the cyanate ion is provided by potassium cyanate (KOCN). The reaction conditions, such as reaction time, and temperature may vary without departing from the scope of the invention. Generally speaking, the peptide hydrazide will be treated with a molar excess of KOCN in the presence of an acid in a protic solvent or a combination of aprotic and protic solvents. The amount of peptide hydrazide to the amount of potassium cyanate may be expressed as a molar ratio of from about 1:1 to about 1:5. Exemplary reaction parameters are detailed in the examples. In an exemplary embodiment, the deprotected peptide hydrazide is treated with a 1.5 molar excess of KOCN in a 5% solution of acetic acid in water. In another exemplary embodiment, the deprotected peptide hydrazide is treated with a 1.5 molar excess of KOCN in a solution of 5% solution of acetic acid in water and acetonitrile(5:1).

Goserelin is typically analyzed by chromatography, such as reverse phase HPLC or mass spectrometry. As will be appreciated by a skilled artisan the yield and purity can and will vary. The yield will generally range from about 40% to greater than about 90%. More typically, the yield will range from about 60% to greater than about 80%. The purity will generally range from about 65% to greater than about 99% as determined by HPLC.

### DEFINITIONS

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “base” is intended to mean an organic or inorganic substance with a pKa of greater than about 8.

“SPPS” as used herein stands for solid phase peptide synthesis.

“Boc” as used herein stands for tert-butyloxycarbonyl.

“DIC” as used herein stands for diisopropylcarbodiimide.

“DIEA” as used herein stands for diisopropylethylamine.

“DCM” as used herein stands for dichloromethane.

“DMF” as used herein stands for dimethylformamide.

“Fmoc” as used herein stands for 9-fluorenyl-methoxy-carbonyl.

“HBTU” as used herein refers to 2-(1H-Benzotriazole-1-yl)-1,1,3,3-tetramethyluronium hexafluorophosphate.

“HOST” as used herein stands for 1-hydroxybenzotriazole.

“TIS” as used herein stands for triisopropylsilane.

“NaBH₄” as used herein stands for sodium borohydride.

“NaOH” as used herein stands for sodium hydroxide.

“ON” as used herein stands for overnight.

“TFA” as used herein stands for trifluoroacetic acid.

As various changes could be made in the above compounds, products and methods without departing from the scope of the invention, it is intended that all matter contained in the above description and in the examples given below, shall be interpreted as illustrative and not in a limiting sense.

### Examples

### Example 1

### Boc/Fmoc Synthesis on Merrifield Resin Using Unprotected Amino Acids, and Cleavage of the Peptide by Hydrazine

In this method, peptide synthesis was performed using amino acids with their amine groups protected by a combination of Boc- and Fmoc-protecting groups. Only the side chain of the D.Ser residue was protected. Addition of the azaglycine moiety was performed after peptide synthesis.

Peptide synthesis: Peptide synthesis procedures followed standard procedure described in the art, and were as follows. Boc-Pro-Merrifield resin (Bachem, Calif.) (9 g; Sub.=0.73 mm/g) was deprotected by treating with a solution of 50% TFA in DCM for 3 min, with the treatment repeated for 20 minutes. Boc-Arg(HCl) was then coupled to the resulting H,Pro-Merrifield resin using 5% DIEA in DCM for 4 min, and the coupling repeated. The same procedure was repeated using Boc-Leu to produce H,Leu-Arg(HCl)-Pro-Merrifield resin. All Boc-protected amino acids were used at a 1.5 molar excess. All the additional amino acids in the peptide were coupled using Fmoc-protected amino acids and the DIC/HOBt coupling method using a 1.5 molar excess of Fmoc-protected amino acids. To remove the Fmoc group after each coupling of Fmoc-amino acid, the nascent peptide was treated with 20% piperidine. The weight of the peptide-resin after peptide synthesis was 18.3 g, which is 87.98% of the theoretical yield of 20.8 g. The net gain in weight was 9.3 g, which is 78.8% of the theoretical yield of 11.8 g.

Release of peptide, and production of goserelin: The peptide was released from the solid support with hydrazine to produce peptide-hydrazide. The peptide-resin (2 g) was treated with 5 ml hydrazine hydrate (H2NNH2.H2O) in 8 ml DMF for 46 hours at room temperature. The gummy mass of the product was assayed by HPLC to show the presence of three major peaks: 26.97% @ 2.31 min, 4.8% @ 9.22 min, and 14.48% @ 21.39 min. The peptide-hydrazide was then converted into goserelin by reacting with KOCN and a 5% solution of acetic acid, followed by lyophilization. HPLC analysis revealed four major peaks: 20.05% @ 20.56 min (goserelin product), 6.5% @ 10.97 min, 2.9% © 12.3 min, and 12.7% @ 20.04

This method was repeated using 2 g peptide-resin, 6 ml hydrazine, and 8 ml DMF for a reaction time of 70 hrs at room temperature to yield 3.5 g of peptide-hydrazide. HPLC results showed three major peaks: 33.65% @ 2.32 min, 20.18% @ 9.23 minutes, and 9.48% @ 21.79 minutes. The peptide hydrazide was converted to goserlin by dissolving in 100 ml of AcOH+H2O (5:95) and treating with a 1.5 molar excess of KOCN in water, while stirring for an hour. The solution was lyophilized to produce goserelin. HPLC results were 6% @ 21.5 min (goserelin), 40% @2.35 min, 16% © 9.35 min. Standard peak was @ 21.75 min (98.74) and co-injection peak @ 21.59 min (15.5%).

### Example 2

### Boc/Fmoc Synthesis on Merrifield Resin Using Unprotected Amino Acids and Cleavage of the Peptide by Semicarbazide

The peptide-solid support synthesized in Example 1 was also treated with semicarbazide in an attempt to generate goserelin using an alternate method. 2 g of the peptide-solid support from Example 1 was contacted with a 15 fold molar excess of semicarbazide.HCl and a 15 fold molar excess of DIEA in 12 ml of DMF reaction solvent, for 46 hours at room temperature. The reaction yielded an oil, but HPLC analysis showed that no goserelin was produced. There were two HPLC peaks: >71.03% @ 9.25min, and >14.13% @ 7.65 min. It was concluded that the peptide was not released from the solid support because there was no reaction with semicarbazide.

### Example 3

### Boc/Fmoc Synthesis on Merrifield Resin Using Protected Amino Acids and Cleavage of the Peptide by Hydrazine

In this method, which consisted of 4 main steps, peptide synthesis was performed using Boc- and Fmoc-protected amino acids. In addition to the side chain of the D.Ser residue being protected by the tBu group, the following Fmoc-amino acids with protected side chain groups were also used: Fmoc-Tyr(2-Cl-trt), Fmoc-Ser(Trt), and Fmoc-His(Trt). Peptide synthesis was followed by deprotection of all side chain protecting groups except that of D.Ser, and addition of the azaglycine moiety.

### Step 1, Peptide Synthesis:

Peptide synthesis followed standard procedure described in the art, and were as follows. Boc-Pro-Merrifield resin (Bachem, Calif.) (9 g, Sub.=0.73 mm/g) was deprotected by treating with a solution of 50% TFA in DCM for 3 minutes. Treatment was repeated for 20 minutes. The deprotected Pro-Merrifield resin was neutralized using 5% DIEA in DCM twice for 4 minutes each. Boc-Arg(HCl) was then activated, and coupled to the proline amino acid on the solid support. The procedure was repeated using Boc-Leu to produce H.Leu-Arg(HCl)-Pro-Merrifield resin. All the additional amino acids in the peptide sequences were coupled using Fmoc-amino acids and the DIC/HOBt coupling method to couple D.Ser(tBu), Tyr(2-Cl-Trt), Ser(Trt), Trp, His(Trt) and p.Glu. All Boc- or Fmoc-protected amino acids were used at a 1.5 molar excess of the solid support, in 1:1 DCM:DMF solvent. 20% piperidine was used to remove the Fmoc group after each addition of Fmoc-amino acid. The weight of the peptide-resin after peptide synthesis was 13.71 g, which is 86.8% of the theoretical yield of 17.8 g. The net gain in weight was 4.71 g, which is 69.3% of the theoretical gain of 6.8 g.

In a second attempt, 5 g of Boc-Pro-Merrifield resin was used and the same procedure repeated as described above. The weight of the peptide-resin after peptide synthesis was 8.82 g, which is 74% of the theoretical yield of 11.92 g. The net gain in wt was 3.82 g, which is 55.2% of the theoretical yield of 6.92 g.

### Step 2, Release of Peptide:

The protected peptide-solid support was first cleaved with hydrazine to produce peptide-hydrazide. Four attempts using various conditions were performed and they are described below.

Prep (A): 1.91 g of the peptide-resin from the first step was treated with 5 ml hydrazine hydrate (H2NNH2.H2O) in 8 ml DMF for 48 hours at room temperature. The reaction product was filtered and washed with DMF and DCM, evaporated to dryness, and then treated with water to isolate the peptide-hydrazide as a colorless solid. The reaction yielded 0.9 g, which is 75,63% of a theoretical yield of 1.19 g. The product was tested by HPLC to show a product peak of 37.9% @ 29.21 min.

Prep (B): The same procedure was repeated using 5 g of peptide-resin to yield 2.88 g of peptide hydrazide, which is 90.6% of the theoretical yield, The product was tested by HPLC to show a product peak 37.34% @ 27.11 min.

Prep (C): 2 g of the peptide-resin from the first step was treated with 6 ml hydrazine hydrate (H2NNH2.H2O) in 8 ml DMF for 70 hours at room temperature. The reaction product was filtered and washed with DMF and DCM, evaporated to dryness, and then treated with ether to isolate the peptide-hydrazide as a hygroscopic solid. The reaction yielded 4.04 g. The product was tested by HPLC to show a major peak of 16.24% @ 28.04 min. The major peak was crystallized with water to get a non-hygroscopic solid. The yield was 0.97 g, which is 77.6% of a theoretical yield of 1.25 g. The product was tested by HPLC to show a major peak of 20.3% @ 29.24 min, and another peak of 15.6% @ 31.46 min.

Prep (C): 1 g of the peptide-resin from the first step was treated with 3 ml hydrazine hydrate in 6 ml DMF/MeOH (1:1) for 48 hours at room temperature. The reaction product was filtered and washed with DMF once and MeOH once, then evaporated to dryness to yield an oil. The oil was treated with ether to isolate the peptide-hydrazide as a solid. The yield was 0.32 g with a major HPLC peak>18.35% @ 30.28 min.

### Step 3, Deprotection:

The protected peptide hydrazide was deprotected to produce peptide hydrazide. Three attempts using various conditions were performed and they are described below.

Prep (A): 1.9 g of the protected peptide hydrazide from Step 2 was deprotected by treating with 15 ml of 3% TFA/DCM and 3% TIS for 1.5 hr and was evaporated to dryness. The deprotecte product was then isolated with ether to yield 1.64 g of colorless solid deprotected peptide-hydrazide.

Prep (B): 0.97 g of the protected peptide hydrazide was treated with 15 ml of 3% TFA/DCM and 3% TIS for 1.5 hr to yield 0.79 g, which is 102.6% of a theoretical yield of 0.77g of product.

Prep (C): 1.4 g of the protected peptide was treated with 26.2 ml of 4% TFA/DCM and 5% TIS (1 ml+24 ml+1.25 ml) at room temperature for 1.5 hr and was then evaporated to dryness. This was followed by isolating the deprotected product with ether to yield 0.3 g of colorless solid deprotected peptide-hydrazide

### Step 4, Production of Goserelin:

Three attempts at production of goserelin were performed using various conditions and they are described below.

Prep (A); 0.74 g of the deprotected peptide-hydrazide from Step 3 was converted into goserelin by dissolving in 30 ml 5% solution of acetic acid and treating with 1.5 molar excess of KOCN while stirring for 1-2 hr. The solution was then lyophilized to yield 0.53 g of an off-white solid, which is 69.7% of the theoretical yield. HPLC analysis showed a product peak of 14% goserelin. The same procedure was repeated for 0.9 g deprotected peptide hydrazide.

Prep (B): The deprotected peptide was converted to goserelin as described in Example 1, for an HPLC yield of 9%.

Prep (C): 0.3 g of deprotected peptide hydrazide was dissolved in 50 ml of a 5% solution of acetic acid and treated with 200mg of KOCN in water. After stirring for 1 hr, the solution was lyophilized into a colorless solid. However, HPLC analysis revealed no product. The syrup from the above reaction was then treated with KOCN/H2O and worked up as described above. The conversion of protected peptide hydrazide into peptide hydrazide into goserelin yielded goserelin at only 7.8% yield.

### Example 4

### Boc/Fmoc Synthesis on Merrifield Resin Using Protected Amino Acids and Cleavage of the Peptide by Semicarbazide

The peptide-solid support synthesized in Example 3 was also treated with semicarbazide in an attempt to generate goserelin using an alternate method. 2 g of the peptide-resin was treated with a 15 molar excess of semicarbazide HCl and a 15 molar excess of DIEA. The reaction was performed in 12 ml DMF solvent for 48 hours at room temperature. However, HPLC analysis revealed that the peptide was not cleaved from the resin and there was no reaction with semicarbazide.

### Example 5

### Boc/Fmoc Synthesis on Merrifield Resin Using Protected Amino Acids and Cleavage of the Peptide by Transesterification

The peptide-solid support synthesized in Example 3 was also released from the resin by transesterification with a (95:5) methanol (MeOH) and Triethyl amine (Et3N) solution to produce peptide methyl ester, then reacted with hydrazine to produce peptide-hydrazide. 2 g of protected peptide-resin was stirred with 20 ml of MeOH/Et3N (95:5) at room temperature for 2 days and filtered. The filtrate evaporated to dryness, and the residue was treated with ether and filtered to yield 0.01 g of peptide-methyl ester. The cleaved resin was again stirred with 10 ml of MeOH/Et3N at (90:10) for 2 days, filtered, evaporated to dryness and treated with ether to get additional 0.01 g of peptide-methyl ester. HPLC analysis revealed several peaks (˜14) with a main peak>8,9% @ 26.18 min, and was therefore found to be not effective, and was not used to produce goserelin.

### Example 6

### Fmoc Synthesis on Hydrazine-CTC Resin Using Protected Amino Acids and Cleavage of the Peptide by Weak Acid (2% TFA in DCM Containing 5% TIS)

In this synthesis, 5 g of Hydrazine-2-Cl-Trt-resin (sub.=1.1 mm/g) and 1.5 fold molar ratio of Fmoc-amino acids were used following standard solid phase peptide synthesis techniques. The Fmoc-amino acids were: Fmoc-Pro, Fmoc-Arg(HCl), Fmoc-Leu, Fmoc-D.Ser(tBu), Fmoc-Tyr(2-Cl-Trt), Fmoc-Ser(Trt), Fmoc-Trp, Fmoc-His(Mtt) and pGlu. HOBT/HBTU/DIEA activation was used for coupling the Fmoc-Pro amino acid to the hydrazine-resin. Additional amino acids are added following the protocol in Table 1.

TABLE 1
Peptide Synthesis Protocol.

| Step No. | Reagents/Solvents* | Times × Minutes |
|---|---|---|
| 1 | DMF Wash | 1 × 3 minutes |
| 2 | 20% Piperidine in DMF | 2 × 20 minutes |
| 3 | DMF Wash | 2 × 3 minutes |
| 4 | IPA wash | 1 × 3 minutes |
| 5 | DMF Wash | 3 × 3 minutes (negative chloraniel |
| | | test) |
| 7 | Coupling | |
| 8 | DMF wash | 1 × 3 minutes |
| 9 | IPA wash | 1 × 3 minutes |
| 10 | DMF wash | 1 × 3 minutes |

There was a net 1.07 g loss in weight instead of the weight gain expected. Therefore the synthesis was unsuccessful.

### Example 7

### Fmoc Synthesis on CTC Resin Using Protected Amino Acids

In this synthesis, CTC resin is used to synthesize a protected peptide acid, which is then reacted with hydrazine to produce protected hydrazide-peptide. The protected peptide hydrazide is partially deprotected in a manner such that the side chains of tyrosine, serine, and histidine are deprotected, but the side chain of D-serine remains protected with tertiary butyl alkyl. The partially deprotected peptide hydrazide is in turn reacted with KOCN to produce Goserelin.

25 g of CTC resin (sub.=1.45 mm/g) is first loaded with Fmoc-Pro using DIEA in DCM for 2 hours. Any unreacted amines were capped with a 9:1 solution of Methanol+DIEA for 20 minutes. DBU analysis revealed a substitution rate=0.81 mm/g. The Fmoc-Pro-resin was de-protected with 20% piperidine/DMF twice for 20 minutes each, and washed with DMF (2×), IPA (1×), DMF (1×) and DCM (1×), to get 31.58 g H.Pro-resin. The same procedure was repeated on a 15 g scale to produce Fmoc-Pro-resin at sub=0.85 mm/g and 17.74 g H.Pro-resin.

### Step 1: Peptide Synthesis

1.5 fold molar ratio of Fmoc-amino acids were used following standard solid phase peptide synthesis techniques using 10 g of H.Pro-resin. The Fmoc-amino acids were: Fmoc-Arg, Fmoc-Leu, Fmoc-D.Ser(tBu), Fmoc-Tyr(2-Cl-Trt), Fmoc-Ser(Trt), Fmoc-Trp, Fmoc-His(Mtt) and pGlu. Amino acids were added following the protocol in Table 1, and 1.5 molar excess of HOBT.H2O and DIC was used. Fmoc-Arg was coupled twice and acetylated, Fmoc-D.Ser(tBu), Fmoc-Tyr(2-Cl-Trt), Fmoc Ser(Trt), Fmoc-Trp and Fmoc-His(Mtt) were coupled once and acetylated, whereas, p.Glu was coupled once and was not acetylated. Peptide-resin yield was 6.69 g, for a net weight loss of 3.31 g.

The peptide synthesis procedure was repeated using 17.74 g resin (syb=0.85 mm/g). Peptide-resin yield was 16.21 g, for a net weight loss of 1.53 g.

### Step 2: Cleavage of Peptide

A number of cleavage methods were attempted and are described below:

### Method (A): With AcOH+TFE+DCM (2:2:6), RT/2 hrs.

2 g of protected peptide-resin was added to 20 ml of AcOH+TFE+DCM (2:2:6) for 2 hrs, then filtered and washed with DCM, TFE, DCM (2× each) and evaporated to dryness. Water was added, filtered, washed with water (2×), and dried to get 0.09 g colorless solid. HPLC analysis using 30-90% B/40min and 90-100% B/5min revealed >25.03% peak product @ 27.32 min out of 5 major peaks. HPLC analysis using 40-90% B/40 min, 90% B/10 min and 90-100% B/2 min revealed >23.3% peak product @ 25.73 min out of 4 major peaks.

Method (B): Cleave with 1% TFA/DCM

2 g of protected peptide-resin was swelled in DCM for 10 min, drained, and cleaved with 1% TFA/DCM (8 times/2 min each). The resin was washed with DCM, MeOH, DCM 92× each) and then evaporated to dryness. Water was then added to the precipitate, filtered and washed two more times with water and dried to get 0.08 g solid. HPLC analysis revealed approximately 8-10 peaks>13% @ 11.32 min, 11.8% @ 16.48 min, >10% © 34.09 min using 40-90% B/40 min, 90% B/10 min, 90-100% B/2 min

Method (C): Cleave with TFE+DCM (3:7), RT/2 hrs (Preferred Method)

1 g of protected peptide-resin was stirred with 10 ml TFE+DCM (3:7) for 2 hrs and was filtered followed by washing with 10 ml of TFE+DCM(3:7), DCM (2×) and evaporated the filtrate to dryness. Water was added to get greenish blue solid which was filtered, washed with water (2×) and dried to get 0.04 g solid. HPLC analysis revealed >32.5% @ 27.33 min and 3 major peaks using 30-90% B/40 min, 90-100% B/5 min.

Method (D): Cleave with 0.1N HCl/DMF+10% TFE.

1 g of protected peptide-resin was stirred with 10 ml 0.1N HCl/DMF+10% TFE at room temperature for 4 hrs. The resin was then filtered, washed with DMF and the filtrate treated with water and kept at 0° C. a few hours and filtered to get 0.01 g of solid. HPLC analysis showed three major peaks with the product peak showing at 24.55% @ 34.68 min.

Method (E): Cleave with 0.1N HCl/THF+10% TFE, RT/4 hrs

1 g of protected peptide-resin was stirred with 10 ml 0.1N HCl/THF+10% TFE at room temperature for 4 hrs. The resin was filtered, washed with THF one time. The filtrate was evaporated to dryness. It was treated with water and filtered to yield 0.06 g of solid. HPLC>17.63% © 25.63 min (of 6 major peaks). HPLC analysis showed six major peaks with the product peak showing at 17.63% @ 25.63 min.

As the results above show, the cleavage method described in Method C produced the best HPLC yields. Therefore, a large scale cleavage of the protected peptide using TFE+DCM (3:7) was performed to prepare for the next step. 12.4 g of protected peptide-resin was stirred with TFE+DCM (3:7) (100 ml) for 2 hrs, filtered and washed with TFE+DCM (3:7) twice, MeOH (twice) and the filtrate was evaporated to dryness to produce an oil. The oil washed with ether, filtered, washed with ether again and dried to yield 0.82 g of colorless solid.

Step 3: Conversion of Peptide Acid into Peptide Hydrazide:

A solution of 0.4 g (0.0002 mole) protected peptide acid prepared using the cleavage method described in Prep C, 3.6 molar equivalents of HOBT.H2O and 4.8 molar equivalents of DIEA in DMF were stirred at 0° C., then 1.2 molar equivalents of HBTU was added and stirring was continued at 0° C. for 20 min. 4 molar equivalents of anhydrous hydrazine were added and the solution was stirred overnight. The solution was then evaporated to dryness, and water was added. The solid was filtered, washed with water twice, washed with a solution of 5% NaHCO3 three times, water twice and dried to get 0.26 g of colorless solid, 65% of the theoretical yield of 0.4 g.

Step 4: Deprotection of Protected Peptide Hydrazide with 2% TFA/DCM+5% TIS

Protected peptide hydrazide (0.2 g) from step 3 above was stirred with 10 ml of 2% TFA/DCM+5% TIS at room temperature for 1.5 hrs and then evaporated to dryness. It was treated with ether, filtered and dried to yield 0.15 g of colorless solid, which is 121.9% of the theoretical yield of 0.123 g.

### Step 5: Preparation of Goserelin

0.15 g of the deprotected peptide hydrazide was dissolved in 20 ml of 5% AcOH/H2O and 5 ml of acetonitrile (ACN, 12.6 fold), and stirred at RT. A 1.5 fold molar equivalent KOCN (0.1 g) was added and stirred for an hour then lyophilized it to get crude goserelin with HPLC>3.16% @ 21.48 min. Presence of the product was confirmed by coinjection with reference compound.

### Example 8

### Synthesis Using a Combination of SPPS and SP Strategy

In this synthesis, CTC resin was used to synthesize a protected peptide-resin of the sequence p.Glu-His(Trt)-Trp-Ser(Trt)-Tyr(2-Cl-Trt)-D.Ser(tBu)-Leu-resin, which was then fused with the activated dipeptide TFA.Arg(HCl)-Pro-OMe to produce p.Glu-H is(Trt)-Trp-Ser(Trt)-Tyr(2-Cl-Trt)-D.Ser(tBu)-Leu-Arg(HCl)-Pro-OMe. This peptide was then reacted with hydrazine to produce protected hydrazide-peptide. The peptide hydrazide was then deprotected and reacted with KOCN to produce Goserelin.

### Step 1: Synthesis of the Partial Peptide Acid:

CTC resin (18 g, Sub.=0.9 mm/g) was first loaded with Fmoc-Leu using 1.2 molar equivalents of DIEA in DCM for 2 hours. The resin was then capped with 50 ml MeOH+DIEA (9:1) for 30 min. DBU analysis showed a substitution rate=0.253 mm/g. The resin was deprotected to yield 7.6 g of H.Leu-resin. Additional Fmoc-amino acids were added using standard peptide synthesis techniques and the amino acids with protected side chains as described in the examples above. The synthesis yielded 25.71 g, which is 99.6% of the 25.82 g theoretical yield of protected peptide-resin.

20 g of protected peptide-resin was cleaved from the resin by treatment with 200 ml TFE+DCM (3:7) for 2 hrs to produce 7.94 (64.08%) of a theoretical peptide acid yield of 12.39 g colorless solid. HPLC analysis revealed a major peak of >79.02% © 34.58 min under the following conditions: 30-90% B/40 min; 90-100% B/2 min; 30% B/10 min. Protected peptide-acid was also synthesized using an automated synthesizer starting with 4 g of H.Leu-resin to yield 6.55 g peptide-resin (114.5% of the theoretical yield of 5.7 g. The peptide acid was cleaved with TFE+DCM (3:7) (70 ml) for 2 hours as described above, to yield 2.24 g peptide acid, or 130.23% of a theoretical yield of 1.72 g. HPLC analysis revealed a major peak of >62.86% @35.06min, confirmed by coinjection with reference compound.

### Step 2: Preparation of Arg(HCl)-Pro-OMe:

Two methods were attempted and are described below:

### Method (A): The MA(IBCF) Method.

Standard solution phase peptide synthesis techniques were used to synthesize the dipeptide, using 12.4 g Boc-Arg(HCl) (0.04 mole), 4.84 ml N-methylmorpholine (NMM) (0.044 mole), 5.71 ml isobutylchloroformate (IBCF, 0.044 mole) and 6.61 g of H.Pro-Ome.HCL (0.04 mole) and 4.4 ml (0.04 mole) NMM. The reaction yielded 25.898 g (153.4%) of the theoretical yield of 16.89 g of the dipeptide oil.

### Method (B): The MA/HOBT Method:

The dipeptide was also prepared using the MA/HOBT method using 12.4 g (0.04 mole) of Boc-Arg(HCl), 4.84 ml (0.044 mole) NMM, 5.71 ml (0.044 mole) of IBCF, 0.04 mole of HOBT and 0.04 mole of H.Pro-OMe to yield 20.5 g (121.4%) of the theoretical yield of 16.88 g of the dipeptide oil.

Step 3: Deprotection of the Dipeptide into TFA.Arq(HCl)-Pro-OMe:

25.89 g of the dipeptide oil was deprotected with 50% TFA/DCM to yield 31.4 g

### Step 4: Preparation of Nonapeptide

3.37 g (0.002 mole) of peptide acid of Step 1 was reacted with a six fold molar excess of the dipeptide TFA.Arg(HCl)-Pro-OMe (7.8 g) of Step 3 using 1.1 g HOBT.H2O, 1.7 ml DIEA (0.0096 mole), and 0.91 g (0.0024 mole) of HBTU. The reaction yielded 3.91 g of a colorless solid (96.5%) of a theoretical yield of 4.02 g. HPLC analysis revealed a product peak of >11.13% @ 28.87min and 6 other major peaks.

Step 5: Conversion of the Protected Nanopeptide-OMe into protected Peptide-Hydrazide.

1.27 g of the nonapeptide (0.63 mm) from Step 4 was dissolved in 15 ml of DMF and MeOH (1:2) and was treated with 1.1 ml of anhydrous hydrazide with stirring. The nonapeptide-hydrazide product was isolated as described above to yield 1.27 g, or 101.6% of a theoretical yield of 1.25 g.

Step 6: Deprotection with 3% TFA/DCM+3% TIS at room temperature for 1.5 hr.

1 g of peptide hydrazide from Step 5 was deprotected as described in Example X and worked up to yield 0.82 g of deprotected peptide hydrazide, equivalent to 133.7% of the theoretical yield of 0.613 g.

Step 7: Conversion into Goserelin.

0.82 g of deprotected peptide hydrazide from Step 6 was stirred with 50 ml of 5% AcOH/H2O containing ACN and 61 mg of KOCN. It was filtered and lyophilized to yield 0.75 g (118.1%) of a solid. However, HPLC analysis showed that there was almost no goserelin peak @ 21.92 min.

### Example 9

### Synthesis Using a Combination of SPPS and SP Strategy, and Conversion of Nonapeptide-OMe to Goserelin Using Semicarbazide

The nonapeptide-OMe synthesized in Step 5 of Example 8 was converted into goserelin with semicarbazide. Protected nonapeptide-OMe (1.27 g) was reacted with a 20 fold molar excess of semicarbazide.HCl (1.4 g) in the presence of a 20 fold molar excess of DIEA in DMF. The product was isolated as a solid with a yield of 1.29 g, or 100.8% of a theoretical yield of 1.28 g. HPLC analysis showed several peaks. 1 g of the product was deprotected by stirring with 16 ml of 3% TFA/DCM+3% TIS for 1.5 hr, and was evaporated to dryness. It was then precipitated with ether as a colorless solid to yield 0.8 g, or 126% of a theoretical yield of 0.635 g. This synthesis was unsuccessful, as HPLC analysis showed no product peak.

### Example 10

### Synthesis Using a Combination of SPPS and SP Strategy

In this synthesis, Sieberamide resin was used. Azaglycine was added to the resin using two methods. The rest of the peptide was then synthesized using protected amino acids and standard Fmoc chemistry. Simultaneously deprotecting and releasing the peptide from the resin using an acid treatment then produces Goserelin.

Step 1: Providing a Solid Support Coupled with Azaglycine

Two methods were attempted and are described below:

### First Method: Preparation of Fmoc-azagly-OSu:

Fmoc-OSu (13.05 g; 0.04 mole) in 200 ml ACN was added to an equimolar amount of hydrazine ((1.3 ml; 0.04 mole) to produce Fmoc-hydrazine at a yield 7.65 g, or 69.32% of a theoretical yield of 10.17 g colorless solid. A second attempt produced a total yield of 8.75 g, or 86.04% of a theoretical yield of 10.17 g. 7.05 g of the Fmoc-hydrazine was then turned into Fmoc-azagly-OSu by reacting with 7.75 g of disuccinimidocarbonte (DSC) in 130 ml of ACN, to yield 9.86 g, or 90.71% of a theoretical yield of 10.87 g.

The Fmoc-azagly-OSu was used to couple Fmoc-azagly onto the solid support. 6.35 g (Sub.=0.63 mm/g) Fmoc-Sieberamide solid support was first deprotected, and then coupled to a two fold molar excess of Fmoc-azagly-OSU in DMF to produce a resin coupled with azaglycine at a substitution rate of 0.35 to 0.6 mm/g.

### Second Method: Load of SA Resin by Triphosgene and Fmoc-azaglycine:

200-300 mg of Sieberamide resin was stirred in DMF, DIEA and triphosgene. Fmoc-hydrazine was prepared as described above and added to the solution. The reaction was then filtered and washed with DMF, MeOH, DCM and dried to produce Sieberamide resin couple to azagly with a substitution rate of 0.03 mm/g to 0.15 mm/g.

In another iteration, 1.0 g Sieberamide resin having free amine liberated (sub=0.63 mm/g) was stirred with 10 ml of DCM and was treated with 3 molar equivalents of triphosgene at room temperature in the presence of 3 molar equivalents of DIEA. Afterward, 2.5 equivalents of Fmoc-hydrazine in 10 ml DMF was added and the mixture was agitated for 24-48 hrs. Substitution of loaded Fmoc-azagly was determined to be 0.15 mm/g

### Step 2: Peptide Synthesis:

The following Fmoc-protected amino acids were used to synthesize the peptide using standard solid phase peptide synthesis techniques and HOBT, DIC in DMF:DCM (3:1): Fmoc-Pro, Fmoc-Arg, Fmoc-Leu, Fmoc-D.Ser(tBu), Fmoc-Tyr(2-Cl-Trt), Fmoc-Trp, Fmoc-His(Trt), and p.Glu.

### Step 3: Production of Goserelin by Simultaneous Removal of Side Chain Protecting Groups and Cleavage Form Resin.

Multiple conditions were attempted for this step:

Method 1: 3% TFA/DCM+3% TIS for 1.5 hours:

Protected peptide-resin (2 g) was treated with 20 ml reagent for 1.5 hr. Filtrate was evaporated and the residue was treated with ether to yield 0.26 g of the product, which was 37.14% of the theoretical yield of 0.7 g. HPLC analysis revealed a product peak of >1.03% @ 21.78 min. Other major peaks were >24.85% @ 27.28 min; 17.86% @ 28.09 min.

Method 2: 8% TFA/DCM+5% TIS for 1.5 hours:

Protected peptide-resin (1.4 g) was treated with 20 ml reagent for 1.5 hr. Filtrate was evaporated to dryness, precipitated with ether to yield 0.39 g of the product, which was 55.71% of the theoretical yield of 0.7 g HPLC>25.2% @ 21.70 min. Other major peaks>14.92% @ 22.66 min; 17.86% @ 28.09 min. Ref compound was at 82.91% @ 21.62 min.

This method was repeated using 1 g of peptide-SAR (0.0003 mole), and 10 ml 8% TFA/DCM+5% TIS, and stirred for 1.5 hours. Filtrate was evaporated to dryness, treated with ether and filtered to yield 0.26 g of product at 74.3% of the theoretical yield of 0.35 g. HPLC>23.55% @ 22.31 min; 16.34% @ 23.26 min.

Method 3: with 10% TFA/DCM+5% TIS for 1.5 hours:

Protected peptide-resin (1 g) was treated with 10 ml reagent for 1.5 hr, Filtrate was evaporated to dryness, precipitated with ether to yield 0.22 g of the product, which was 62.86% of the theoretical yield of 0.35 g HPLC>23.95% @ 22.28 min; >19.75% @ 23.24 min.

Method 4: with 15% TFA/DCM+5% TIS for 1.5 hours:

Protected peptide-resin (0.8 g) was treated with reagent for 1.5 hr to yield 0.08 g of the product. HPLC>13.46% @ 21.4 min; >21.78% @ 15.37 min.

Conclusion: 8% TFA/DCM+5% TIS for 1.5 hours was found to be the most suitable conditions for the release and deprotection of goserelin from the Sieberamide resin.

What is claimed is:

1. A process for solid phase synthesis of goserelin, the process comprising:
(a) providing a solid support coupled with azaglycine;
(b) activating the carboxy group of a proline residue that has its amine protected by a Fmoc group or a Boo group, followed by coupling the proline residue to the azaglycine residue on the solid support of (a), and treatment of the solid support with an agent to deprotect the amine group of the proline residue;
(c) activating the carboxy group of an arginine residue that has its amine protected by a Fmoc group or a Boc group, followed by coupling the arginine residue to the proline residue on the solid support of (b), and treatment of the solid support with an agent to deprotect the amine group of the arginine residue;
(d) activating the carboxy group of a leucine residue that has its amine protected by a Fmoc group or Boc group, followed by coupling the leucine residue to the arginine residue on the solid support of (c), and treatment of the solid support with an agent to deprotect the amine group of the leucine residue;
(e) activating the carboxy group of a D-serine residue that has its amine protected by a Fmoc group and its side chain protected by a tertiary butyl alkyl group, followed by coupling the D-serine residue to the leucine residue on the solid support of (d), and treatment of the solid support with a base to deprotect the amine group of the D-serine residue;
(f) activating the carboxy group of a tyrosine residue that has its amine protected by a Fmoc group and its side chain protected by an acid labile group, followed by coupling the tyrosine residue to the D-serine residue on the solid support of (e), and treatment of the solid support with a base to deprotect the amine group of the tyrosine residue;
(g) activating the carboxy group of a serine residue that has its amine protected by a Fmoc group and its side chain protected by an acid labile group, followed by coupling the serine residue to the tyrosine residue on the solid support of (f), and treatment of the solid support with a base to deprotect the amine group of the serine residue;
(h) activating the carboxy group of a tryptophan residue that has its amine protected by a Fmoc group, followed by coupling the tryptophan residue to the serine residue on the solid support of (g), and treatment of the solid support with a base to deprotect the amine group of the tryptophan residue;
(i) activating the carboxy group of a histidine residue that has its amine protected by a Fmoc group and its side chain protected by an acid labile group, followed by coupling the histidine residue to the to the tryptophan residue on the solid support of (h), and treatment of the solid support with a base to deprotect the amine group of the histidine residue;
(j) activating the carboxy group of a pyroglutamic acid residue, followed by coupling the pyroglutamic acid residue to the histidine residue on the solid support of (i); and
(k) simultaneously contacting the solid support of (j) with an acid in a manner such that goserelin is released from the solid support and the side chains of tyrosine, serine, and histidine are deprotected, but the side chain of D-serine remains protected with tertiary butyl alkyl.

2. The process of claim 1, wherein the solid support is chosen from NovaSyn® TGR resin, Rink amide resin, Rink amid MBHA resin, Rink amide AM resin, Rink amide PEGA resin, Rink amide NovaGel® resin, Sieber amide resin, and NovaSyn® TG Sieber resin; the acid liable protecting group is chosen from methyltrityl, methoxytrityl, and trityl; the acid is chosen from trifluoroacetic acid, hydrochloric acid, acetic acid and trifluoroethanol; and the carboxylic acid group of the amino acid residues is activated by contacting the amino acid residue with a compound chosen from HOBt, DCC, DIEA, and DIC.

3. The process of claim 1, wherein the acid comprises trifluoroacetic acid and triisopropylsilane.

4. The process of claim 1, wherein the azaglycine is coupled to the solid support by contacting the solid support with Fmoc-hydrazine and triphosgene or the azaglycine is coupled to the solid support by contacting the solid support with Fmoc-azaglycine-OSu, wherein the Fmoc-azaglycine-OSu is synthesized according to Reaction Scheme 1:

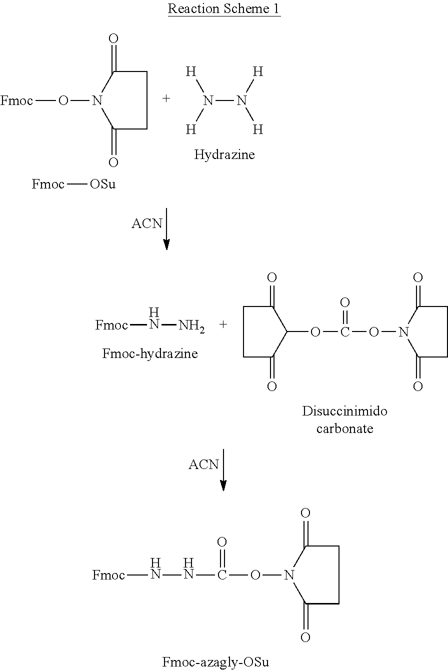

5. The process of claim 1, wherein the amine group of proline, arginine, and leucine are each protected with a Boc group and the agent used for deprotection is chosen from trifluoroacetic acid and hydrogen chloride.

6. The process of claim 1, wherein the amine group of proline, arginine, and leucine are each protected with an Fmoc group and the agent used for deprotection is a base.

7. The process of claim 6, wherein the base used to deprotect the amine group is chosen from piperidine, cyclohexylamine, 1,5-diazabicyclo[5,4,0]undec-5-ene, ethanolamine, pyrrolidine 1,8-diazabicyclo[5.4.0]undec-7-ene, diethylamine, morpholine, and mixtures thereof.

8. The process of claim 1, wherein the yield of goserelin is at least 15%.

9. The process of claim 1, wherein the yield of goserelin is at least 20%.

10. A process for solid phase synthesis of goserelin, the process comprising:
(a) providing a solid support coupled with a proline residue;
(b) activating the carboxy group of an arginine residue that has its amine protected by a Boc group or Fmoc group, followed by coupling the arginine residue to the proline residue on the solid support of (a), and treatment of the solid support with an agent to deprotect the amine group of the arginine residue;
(c) activating the carboxy group of a leucine residue that has its amine protected by a Boc group or Fmoc group, followed by coupling the leucine residue to the arginine residue on the solid support of (b), and treatment of the solid support with an agent to deprotect the amine group of the leucine residue;
(d) activating the carboxy group of a D-serine residue that has its amine protected by a Fmoc group and its side chain protected by a tertiary butyl alkyl group, followed by coupling the D-serine residue to the leucine residue on the solid support of (c), and treatment of the solid support with a base to deprotect the amine group of the D-serine residue;
(e) activating the carboxy group of a tyrosine residue that has its amine protected by a Fmoc group and its side chain protected by an acid labile group, followed by coupling the tyrosine residue to the D-serine residue on the solid support of (d), and treatment of the solid support with a base to deprotect the amine group of the tyrosine residue;
(f) activating the carboxy group of a serine residue that has its amine protected by a Fmoc group and its side chain protected by an acid labile group, followed by coupling the serine residue to the tyrosine residue on the solid support of (e), and treatment of the solid support with a base to deprotect the amine group of the serine residue;
(g) activating the carboxy group of a tryptophan residue that has its amine protected by a Fmoc group, followed by coupling the tryptophan residue to the serine residue on the solid support of (f), and treatment of the solid support with a base to deprotect the amine group of the tryptophan residue;
(h) activating the carboxy group of a histidine residue that has its amine protected by a Fmoc group and its side chain protected by an acid labile group, followed by coupling the histidine residue to the to the tryptophan residue on the solid support of (g), and treatment of the solid support with a base to deprotect the amine group of the histidine residue;
(i) activating the carboxy group of a pyroglutamic acid residue, followed by coupling the pyroglutamic acid residue to the histidine residue on the solid support of (h);
(j) contacting the solid support of (i) with hydrazine in a manner such that a peptide hydrazide is released from the solid support, and the side chains of D-serine, tyrosine, serine, and histidine remain protected;
(k) contacting the peptide hydrazide of (j) with an acid in a manner such that the side chains of tyrosine, serine, and histidine are deprotected, but the side chain of D-serine remains protected with tertiary butyl alkyl; and
(l) contacting the peptide hydrazide from (k) with a cyanate ion source to form goserelin.

11. The process of claim 10, further comprising contacting the solid support of (i) with a cleaving agent in a manner such that a peptide acid is cleaved from the solid support, and the side chains of D-serine, tyrosine, serine, and histidine remain protected, and then contacting the peptide acid with hydrazine in step (j) to form the peptide hydrazine.

12. The process of claim 10, wherein the solid support is chosen from chlorotrityl resins, trityl resins, methyltrityl resins, methoxytrityl resins, NovaSyn® TGT resin, HMPB-AM resin, HMPB-BHA resin, HMPB-MBHA resin, Wang resin, NovaSyn-TGA resin, HMPA-PEGA resin, HMPA-NovaGel resin, PAM resin, and Merrifield resin; the carboxylic acid group of the amino acid residues is activated by contacting the amino acid residue with a compound chosen from HOBt, DCC, DIEA, and DIC; the acid labile protecting group is chosen from methyltrityl, methoxytrityl, and trityl; and the acid is chosen from trifluoroacetic acid, hydrochloric acid, acetic acid and trifluoroethanol.

13. The process of claim 10, wherein the acid comprises trifluoroacetic acid and triisopropylsilan.

14. The process of claim 10, wherein the amine group of proline, arginine, and leucine are each protected with a Boc group and the agent used for deprotection is chosen from trifluoroacetic acid and hydrogen chloride.

15. The process of claim 10, wherein the amine group of proline, arginine, and leucine are each protected with an Fmoc group and the agent used for deprotection is a base.

16. The process of claim 15, wherein the base used to deprotect the amine group is chosen from piperidine, cyclohexylamine, 1,5-diazabicyclo [5,4,0]undec-5-ene, ethanolamine, pyrrolidine 1,8-diazabicyclo[5.4.0]undec-7-ene, diethylamine, morpholine, and mixtures thereof.

17. The process claim 10, wherein the cyanate ion source is chosen from potassium cyanate, methyl cyanate, or sodium cyanate.

18. The process claim 10, wherein the cyanate ion source is potassium cyanate.

19. The process of claim 10, wherein the yield of goserelin is at least 15%.

20. The process of claim 10, wherein the yield of goserelin is at least 20%.
